(19) United States
(12) Patent Application Publication
(10) Pub. No.: US 2008/0152623 A1
(43) Pub. Date: Jun. 26, 2008

(54) Blockade of Airway Hyperresponsiveness and Inflammation in a Murine Model of Asthma by Insulin-Like Growth Factor Binding Protein-3 (Igfbp-3)

(75) Inventors: Yong Chul Lee, Jeonju (KR); Dae Yeol Lee, Jeonju (KR)

Correspondence Address: TROUTMAN SANDERS LLP, 600 PEACHTREE STREET , NE, ATLANTA, GA, 30308, US

(73) Assignee: Biocure Pharma, LLC, Richmond, VA (US)

(21) Appl. No.: 11/884,077

(22) PCT Filed: Feb. 10, 2006
(86) PCT No.: PCT/IB06/01125

§ 371 Date: Aug. 9, 2007

Related U.S. Application Data
(60) Provisional application No. 60/652,023, filed on Feb. 10, 2005.
(60) Provisional application No. 60/698,731, filed on Jul. 13, 2005.

(51) Int. Cl. A61K 48/00; A61P 11/06

(52) U.S. Cl. 424/93.2; 514/44

(57) ABSTRACT

The physiological role of IGFBP-3 in respiratory inflammation and hyperresponsiveness is presently unknown. The present invention is based on the unexpected finding that both wild-type IGFBP-3 and the IGFBP-3 mutant GGG-IGFBP-3 inhibit tissue inflammation and hyperresponsiveness associated with obstructive respiratory disorders such as bronchial asthma. Provided herein are methods of treating obstructive respiratory disorders and various conditions associated with airway hyperresponsiveness, including asthma, by administering recombinant IGFBP-3 or IGFBP-3 mutants or vectors encoding IGFBP-3 or IGFBP-3 mutants.

Rinderknecht 1978b). IGFs are capable of stimulating tissue growth and differentiation by acting in a paracrine, autocrine, and/or endocrine manner (Bach 1995; Marshman 2002). The mitogenic actions of IGFs are mediated largely through the type I IGF receptor (IGFR-I), which is a heterotetrameric, membrane-spanning tyrosine kinase (Nissley 1991; Schumacher 1991). IGFR-I binds both IGF-I and IGF-II with high affinity, and binds insulin with a substantially lower affinity (Marshman 2002).

IGFBPs bind IGF-I and IGF-II with high affinity, but they do not bind insulin. IGFBPs are essential for transporting IGFs, prolonging their half-lives by protecting them from proteolytic degradation, and regulating their availability for interaction with IGFRs (Baxter 1991; Le Roith 2001) In this manner, they modulate the effects of IGF on growth and differentiation by either potentiating or inhibiting IGF activity (Bach 1995). Both the N-terminal and C-terminal domains of the IGFBPs are highly conserved.

Recent research has demonstrated that IGFBPs have unique intrinsic biological activities beyond their ability to interact with IGF, termed the “IGF-independent” actions (Jones 1995; Oh 1998). For example, IGFBP-3 has been shown to exert IGF-independent effects on cell growth and apoptosis (Oh 1993; Longobardi 2003). In order to establish that these biological effects are truly IGF-independent, an IGFBP-3 mutant has been created that has no binding affinity for IGFs. This mutant (referred to as GGG-IGFBP-3, G56G80G81, or simply the GGG mutant) is generated by site-directed mutagenesis of IGFBP-3 residues Ile⁵⁶, Leu⁸⁰, and Leu⁸¹ to Gly⁵⁶, Gly⁸⁰, and Gly⁸¹ (Buckway 2001; Longobardi 2003; Kim 2004). Despite this work, the mechanism underlying the IGF-independent actions of IGFBP-3 has yet to be elucidated.

Bronchial asthma is a chronic inflammatory disease of the airways characterized by airway eosinophilia, goblet cell hyperplasia with mucus hypersecretion, and hyperresponsiveness to inhaled allergens and to nonspecific stimuli (Kay 1991). Eosinophil response appears to be a critical feature in asthma. Eosinophil accumulation and subsequent activation in bronchial tissues play critical roles in the pathophysiology of bronchial asthma (Frigas 1986). Recent studies have suggested that airway inflammation may be perpetuated by bronchial epithelial cells themselves. Epithelial cells have been shown to produce numerous inflammatory mediators, such as platelet activating factor and prostaglandins (Holgate 2000). Bronchial epithelial cells also produce a wide variety of proinflammatory cytokines, such as IL-1, IL-5, IL-6, IL-8, GM-CSF, TNF, MCP-1, and RANTES (Holgate 2000). Production of cytokines and chemoattractants by bronchial epithelial cells in subjects with bronchial asthma and other airway inflammatory diseases appears to contribute to the local accumulation of inflammatory cells.

IGF-I has been identified as a major fibroblast mitogen produced by human airway epithelial cells (Cambrey 1995). Other studies have demonstrated that inhaled corticosteroid reduces lamina reticularis of the basement membrane by suppression of IGF-I expression in bronchial asthma (Hoshino 1998). Significant correlation has been shown between IGF-I expression and both collagen thickening and fibroblast number, suggesting that IGF-I may be involved in the inflammatory process associated with bronchial asthma. With regards to IGFBPs, it has been demonstrated that the IGFBP protease matrix metalloproteinase-1 (MMP-1) is elevated in asthmatic airway smooth muscle cells (Rajah 1999). In addition, IGFBP-2 and -3, both of which are proteolytic substrates of MMP-1, have been shown to be cleaved in asthmatic airway tissue extracts (Rajah 1999). These studies indicate that the IGF system plays an important role in the inflammatory process associated with asthma.

### BRIEF DESCRIPTION OF THE DRAWINGS

FIG. 1: Schematic diagram of the experimental protocol. Mice were sensitized on days 1 and 14 by intraperitoneal injection of OVA emulsified in 1 mg of aluminum hydroxide. On days 21, 22, and 23 after the initial sensitization, the mice were challenged for 30 minutes with an aerosol of 3% (w/v) OVA in saline (or with saline as a control) using an ultrasonic nebulizer. In the case of treatment with Ad vector, it was administered intratracheally two times to each treated animal, once on day 21 (1 hour before the first airway challenge with OVA) and the second time on day 23 (3 hours after the last airway challenge with OVA).

FIG. 2: Effect of WT-AdIGFBP-3 and m-AdIGFBP-3 administration on total and differential cellular components in BAL fluid in OVA-challenged mice. The numbers of each cellular component of BAL fluid from control mice (SAL+SAL), OVA-challenged mice (OVA+SAL), OVA-challenged mice administered with WT-AdIGFBP-3 (OVA+WT-IGFBP-3), OVA-challenged mice administered with m-AdIGFBP-3 (OVA+m-IGFBP-3), and OVA-challenged mice administered with AdLacZ (OVA+AdLacZ) were counted at 72 hours after the last challenge. Bars represent mean ±SEM from six independent experiments. #, P<0.05 vs. SAL+SAL; *, P<0.05 vs. OVA+SAL.

FIG. 3: Effect of WT-AdIGFBP-3 and m-AdIGFBP-3 administration on pathologic changes in lung tissues of OVA-challenged mice. Representative hematoxylin and eosin-stained sections of the lungs. Sampling was performed at 72 hours after the last challenge. A. Control mice (SAL+SAL). B. OVA-challenged mice (OVA+SAL). C. OVA-challenged mice administered with WT-AdIGFBP-3 (OVA+WT-AdIGFBP-3). D. OVA-challenged mice administered with m-AdIGFBP-3 (OVA+m-AdIGFBP-3). E. OVA-challenged mice administered with AdLacZ (OVA+AdLacZ). Bars indicate scale of 50 μm.

FIG. 4: Effect of WT-AdIGFBP-3 and m-AdIGFBP-3 administration on peribronchial and perivascular lung inflammation. Peribronchial, perivascular, and total lung inflammation were measured at 72 hours after the last challenge in control mice (SAL+SAL), OVA-challenged mice (OVA+SAL), OVA-challenged mice administered with WT-AdIGFBP-3 (OVA+WT-IGFBP-3), OVA-challenged mice administered with m-AdIGFBP-3 (OVA+m-IGFBP-3), and OVA-challenged mice administered with AdLacZ (OVA+AdLacZ). Total lung inflammation was defined as the average of the peribronchial and perivascular inflammation scores. Bars represent mean ±SEM from six independent experiments. #, P<0.05 vs. SAL+SAL; *, P<0.05 vs. OVA+SAL.

FIG. 5: Effect of WT-AdIGFBP-3 and m-AdIGFBP-3 administration on IL-4, IL-5, and IL-13 protein expression in lung tissues of OVA-challenged mice. The expression of cytokines IL-4, IL-5, and IL-1 3 was measured at by Western blot at 72 hours after the last challenge in control mice (SAL+SAL), OVA-challenged mice (OVA+SAL), OVA-challenged mice administered with WT-AdIGFBP-3 (OVA+WT-AdIGFBP-3), OVA-challenged mice administered with m-AdIGFBP-3 (OVA+m-AdIGFBP-3), and OVA-challenged mice administered with AdLacZ (OVA+AdLacZ). Results were similar in six independent experiments.

FIG. 6: Effect of WT-AdIGFBP-3 and m-AdIGFBP-3 administration on IL-4, IL-5, and IL-13 levels in BAL fluid of OVA-challenged mice. The level of IL-4, IL-5, and IL-13 was measured by immunoassay 72 hours after the last challenge in control mice (SAL+SAL), OVA-challenged mice (OVA+SAL), OVA-challenged mice administered with WT-AdIGFBP-3 (OVA+WT-IGFBP-3), OVA-challenged mice administered with m-AdIGFBP-3 (OVA+m-IGFBP-3), and OVA-challenged mice administered with AdLacZ (OVA+AdLacZ). Bars represent mean ±SEM from six independent experiments. #, P<0.05 vs. SAL+SAL; *, P<0.05 vs. OVA+SAL.

FIG. 7: Effect of WT-AdIGFBP-3 and m-AdIGFBP-3 administration on TNF-αand IL-1β, protein expression in OVA-challenged mice. A. TNF-α and IL-1β protein expression was measured by Western blotting in lung tissues of OVA-challenged mice. Expression was measured at 72 hours after the last challenge in control mice (SAL+SAL), OVA-challenged mice (OVA+SAL), OVA-challenged mice administered with WT-AdIGFBP-3 (OVA+WT-AdIGFBP-3), OVA-challenged mice administered with m-AdIGFBP-3 (OVA+m-AdIGFBP-3), and OVA-challenged mice administered with AdLacZ (OVA+AdLacZ). Results were similar in six independent experiments. B. TNF-α and IL-1β protein expression was measured by enzyme immunoassay in BAL fluid of OVA-challenged mice. Expression was measured at 72 hours after the last challenge in control mice (SAL+SAL), OVA-challenged mice (OVA+SAL), OVA-challenged mice administered with WT-AdIGFBP-3 (OVA+WT-AdIGFBP-3), OVA-challenged mice administered with m-AdIGFBP-3 (OVA+m-AdIGFBP-3), and OVA-challenged mice administered with AdLacZ (OVA+AdLacZ). Bars represent mean ±SEM from six independent experiments. #, P<0.05 vs. SAL+SAL; *, P<0.05 vs. OVA+SAL.

FIG. 8: Effect of WT-AdIGFBP-3 and m-AdIGFBP-3 administration on VCAM-1 and ICAM-1 protein expression. A. VCAM-1 and ICAM-1 protein expression was measured by Western blotting in lung tissues of OVA-challenged mice. Expression was measured at 72 hours after the last challenge in control mice (SAL+SAL), OVA-challenged mice administered with saline (OVA+SAL), OVA-challenged mice administered with WT-AdIGFBP-3 (OVA+WT-AdIGFBP-3), OVA-challenged mice administered with m-AdIGFBP-3 (OVA+m-AdIGFBP-3), and OVA-challenged mice administered with AdLacZ (OVA+AdLacZ). Results were similar in six independent experiments. B. Western blot results were quantitated by densitometric analysis based on the relative ratio of VCAM-1 or ICAM-1 to actin. The relative ratio of VCAM-1 or ICAM-1 in SAL+SAL mice is arbitrarily presented as one. Data represent mean ±SEM from six independent experiments. #, P<0.05 vs. SAL+SAL; *, P<0.05 vs. OVA+SAL.

FIG. 9: Effect of WT-AdIGFBP-3 and m-AdIGFBP-3 administration on eotaxin and RANTES protein expression. A. Eotaxin and RANTES protein expression was measured by Western blotting in lung tissues of OVA-challenged mice. Expression was measured at 72 hours after the last challenge in control mice (SAL+SAL), OVA-challenged mice (OVA+SAL), OVA-challenged mice administered with WT-AdIGFBP-3 (OVA+WT-AdIGFBP3), OVA-challenged mice administered with m-AdIGFBP-3 (OVA+m-AdIGFBP3), and OVA-challenged mice administered with AdLacZ (OVA+AdLacZ). Results were similar in six independent experiments. B. Eotaxin and RANTES protein expression was measured by enzyme immunoassay in BAL fluid of OVA-challenged mice. Expression was measured at 72 hours after the last challenge in control mice (SAL+SAL), OVA-challenged mice (OVA+SAL), OVA-challenged mice administered with WT-AdIGFBP-3 (OVA+WT-AdIGFBP-3), OVA-challenged mice administered with m-AdIGFBP-3 (OVA+m-AdIGFBP-3), and OVA-challenged mice administered with AdLacZ (OVA+AdLacZ). Bars represent mean ±SEM from six independent experiments. #, P<0.05 vs. SAL+SAL; *, P<0.05 vs. OVA+SAL.

FIG. 10: Effect of WT-AdIGFBP-3 and m-AdIGFBP-3 administration on airway responsiveness in OVA-challenged mice. Airway responsiveness was measured at 72 hours after the last challenge in control mice (SAL+SAL), OVA-challenged mice (OVA+SAL), OVA-challenged mice administered with WT-AdIGFBP-3 (OVA+WT-IGFBP-3), OVA-challenged mice administered with m-AdIGFBP-3 (OVA+m-IGFBP-3), and OVA-challenged mice administered with AdLacZ (OVA+AdLacZ). Airway responsiveness to aerosolized methacholine was measured in unrestrained, conscious mice. Mice were nebulized with saline, then with increasing doses (2.5 to 50 mg/ml) of methacholine for three minutes at a time. Readings of breathing parameters were taken for three minutes after each nebulization, during which Penh values were determined. Data represent mean ±SEM from six independent experiments. #, P<0.05 vs. SAL+SAL; *, P<0.05 vs. OVA+SAL.

FIG. 11: IGFBP-3 expression in lung tissue following OVA challenge. A. IGFBP-3 expression was measured by Western blot for control mice (SAL+SAL), OVA-challenged mice (OVA+SAL), OVA-challenged mice administered with AdIGFBP-3 (OVA+IGFBP-3), and OVA-challenged mice administered with AdLacZ (OVA+AdLacZ). Results were similar in six independent experiments. B. IGFBP-3 expression was measured by Western blot for OVA-challenged mice ((OVA) IGFBP-3) and control mice ((saline) IGFBP-3) at various timepoints following challenge. Results were similar in ten independent experiments.

FIG. 12: IGF-1 expression in BAL fluid following OVA challenge. IGF-1 protein expression was measured by enzyme immunoassay in BAL fluid of OVA-challenged mice. Expression was measured prior to challenge (PRE) and at 1 hour, 6 hours, 24 hours, and 48 hours 72 hours after the last challenge in control mice (SAL) and OVA-challenged mice (OVA). Bars represent mean ±SEM from six independent experiments. #, P<0.05 vs. SAL+SAL; *, P<0.05 vs. Pre. Sensitivity for the assay was 3.5 μg/ml.

FIG. 13: Immunohistochemical analysis of IGFBP-3 protein in lung tissue and tracheal epithelial cells. Dark brown color indicates IGFBP-3-positive cells. Bars indicate scale of 50 μm. A. Lung tissue of control mice. B. Lung tissue of OVA-challenged mice. C. Lung tissue of OVA-challenged mice administered with WT-AdIGFBP-3. D. Lung tissue of OVA-challenged mice administered with AdLacZ. E. Tracheal epithelial cells of control mice. F. Tracheal epithelial cells of OVA-challenged mice. G. Tracheal epithelial cells of OVA-challenged mice administered with WT-AdIGFBP-3. H. Tracheal epithelial cells of OVA-challenged mice administered with AdLacZ.

FIG. 14: Effect of recombinant IGFBP-3 administration on total and differential cellular components in BAL fluid of OVA-challenged mice. The numbers of each cellular component of BAL fluid from control mice (SAL+SAL), OVA-challenged mice (OVA+SAL), OVA-challenged mice administered with 1 μg recombinant IGFBP-3 (OVA+IGFBP3 1 μg), and OVA-challenged mice administered with 10 μg recombinant IGFBP-3 (OVA+IGFBP3 10 μg) were counted at 72 hours after the last challenge. Bars represent mean ±SEM from ten independent experiments. #, P<0.05 vs. SAL+SAL; *, P<0.05 vs. OVA+SAL.

FIG. 15: Effect of recombinant IGFBP-3 administration on airway responsiveness in OVA-challenged mice. Airway responsiveness was measured at 72 hours after the last challenge in control mice (SAL+SAL), OVA-challenged mice (OVA+SAL), OVA-challenged mice administered with 1 μg recombinant IGFBP-3 (OVA+IGFBP3 μg), and OVA-challenged mice administered with 10 μg recombinant IGFBP-3 (OVA+IGFBP3 10 μg). Airway responsiveness to aerosolized methacholine was measured in unrestrained, conscious mice. Mice were nebulized with saline, then with increasing doses (2.5 to 50 mg/ml) of methacholine for three minutes at a time. Readings of breathing parameters were taken for three minutes after each nebulization, during which Penh values were determined. Data represent mean ±SEM from ten independent experiments. #, P<0.05 vs. SAL+SAL; *, P<0.05 vs. OVA+SAL.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

### Abbreviations

The following abbreviations are used herein: Ad, adenovirus; BAL, bronchoalveolar lavage; COPD, chronic obstructive pulmonary diseases; ICAM-1, intercellular adhesion molecule-1; IGF, insulin-like growth factor; IGFBP, insulin-like growth factor binding protein; IGFR, insulin-like growth factor receptor; OVA, ovalbumin; SEM, standard error of the mean; VCAM-1, vascular cell adhesion molecule-1.

### Definitions

The phrase “airway hyperresponsiveness” as used herein refers to increased sensitivity of the airway to a constrictor agonist relative to a normal airway. A constrictor agonist may be a direct agonist, such as histamine, methacholine, citric acid, an allergen, a respiratory virus, or a foreign particle, or an indirect agonist such as exercise or inhalation of cold or dry air.

The phrase “obstructive respiratory disorder” as used herein refers to a condition associated with airway obstruction. This obstruction may arise from airway hyperresponsiveness, inflammation of the respiratory tissue, thickening of the respiratory tissue, or any combination of these. In one embodiment, the affected respiratory tissue is lower respiratory tissue. An obstructive respiratory disorder may be either acute or chronic. Acute disorders include allergic reactions and temporary asthma-like symptoms. Chronic disorders include chronic obstructive pulmonary diseases (COPDs), which may include asthma, cystic fibrosis, chronic bronchitis, emphysema, or bronchiectasis.

The phrase “lower respiratory tissue” as used herein refers to tissue of any organ in the lower respiratory system, including the larynx, trachea, bronchi, bronchioles, and lungs.

The term “vector” as used herein refers to a vehicle into which a genetic element encoding a polypeptide may be operably inserted so as to bring about the expression of that polypeptide. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. A vector may also include or be associated with various materials to aid in its entry into the cell, including but not limited to a viral particle, a liposome, or a protein coating.

The phrase “therapeutically effective amount” as used herein is an amount of a compound that produces a desired therapeutic effect in a subject, such as preventing or treating a target condition or alleviating symptoms associated with the condition. The precise therapeutically effective amount is an amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a compound and adjusting the dosage accordingly. For additional guidance, see Remington: The Science and Practice of Pharmacy 20th Edition, Gennaro, Ed., Williams & Wilkins Pennsylvania, 2000.

The term “treating” as used herein with regards to a condition or disorder may refer to preventing the condition or disorder, slowing the onset or rate of development of the condition or disorder, reducing the risk of developing the condition or disorder, preventing or delaying the development of symptoms associated with the condition or disorder, reducing or ending symptoms associated with the condition or disorder, generating a complete or partial regression of the condition or disorder, or some combination thereof.

The phrase “pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or some combination thereof. Each component of the carrier must be “pharmaceutically acceptable” in that it must be compatible with the other ingredients of the formulation. It must also be suitable for use in contact with any tissues or organs that it may come in contact with, meaning that it must not carry a risk of toxicity, irritation, allergic response, immunogenicity, or any other complication that excessively outweighs its therapeutic benefits.

The term “subject” as used herein refers to any animal, but preferably refers to a mammal or more preferably to a human.

The IGF system is a multicomponent network of molecules that is ubiquitously involved in the regulation of growth, proliferation, and differentiation of a variety of cell types. For example, IGF-I appears to be involved in the inflammatory process associated with bronchial asthma. IGF-1 activity is modulated by IGFBPs. Although the six IGFBPs display high levels of conservation in their C- and N-terminal domains, their expression patterns and properties vary widely. Of the six IGFBPs, IGFBP-3 is the most abundant in serum. IGFBP-3 is known to modulate IGF-1 activity in certain situations, but its pathophysiological role in respiratory inflammation and hyperresponsiveness is unknown. The present invention unexpectedly demonstrates that both wild-type IGFBP-3 and the GGG-IGFBP-3 mutant are inhibitors of the tissue inflammation and hyperresponsiveness associated with obstructive respiratory disorders such as bronchial asthma.

A mouse model for asthma was used to determine the effects of IGFBP-3 on respiratory inflammation and airway hyperresponsiveness. Three adenoviral vectors were generated for these studies. The first, WT-AdIGFBP-3, contained cDNA encoding wild-type IGFBP-3. The second, m-AdIGFBP-3, contained cDNA encoding the GGG-IGFBP-3 mutant. The third, AdLacZ, was used as a control.

Mice were sensitized by intraperitoneal injection of OVA. The adenoviral vectors were administered to the mice intratracheally 21 days after the initial sensitization, and the mice were challenged with OVA on days 21, 22, and 23. 72 hours after the final challenge, BAL was performed and the lungs were removed for analysis. BAL fluid from mice administered with WT-AdIGFBP-3 or m-AdIGFBP-3 displayed significantly reduced numbers of eosinophils, lymphocytes, neutrophils, and total cells. Similar results were obtained when mice were administered with recombinant IGFBP-3. Increased numbers of eosinophils are believed to be associated with many of the tissue changes seen in asthmatic airways, including epithelial damage, thickening of the basement membrane, and the release of mediators with the capacity to cause bronchial smooth muscle contraction and exudation of plasma, resulting in thickening of the airway wall (O'Byrne 2003).

Histological studies of the excised lung tissue revealed that mice treated with WT-AdIGFBP-3 or m-AdIGFBP-3 showed markedly reduced levels of inflammation and inflammatory cell infiltration in both the peribronchiolar and perivascular regions. The histological data also confirmed that mice administered with the adenoviral vectors displayed increased expression of IGFBP-3, confirming the effectiveness of expression from the adenoviral vectors.

Western blot analysis of lung tissue and enzyme immunoassays of BAL fluid revealed that expression of IL-4, IL-5, IL-13, TNF-α, IFN-1β, VCAM-1, ICAM-1, eotaxin, and RANTES increased following challenge with OVA, and that this increase was greatly reduced by administration of WT-AdIGFBP-3 or m-AdIGFBP-3. Western blot analysis also confirmed that endogenous IGFBP-3 levels were significantly reduced following challenge with OVA, while endogenous IGF-1 levels were significantly increased.

Various breathing parameters were measured in response to increasing methacholine concentrations in live, unrestrained mice. The parameters were used to generate a Penh value, and the increase in baseline Penh was used to assess airway responsiveness to methacholine. OVA-challenged mice exhibited airway hyperresponsiveness compared to control mice, as demonstrated by higher Penh values at each methacholine concentration tested. Administration of WT-AdIGFBP-3 or m-AdIGFBP-3 to OVA-challenged mice resulted in a significant decrease in airway hyperresponsiveness, which was indicated by a substantial decrease in Penh values. Similar results were obtained when mice were administered with recombinant IGFBP-3.

These results demonstrate that IGFBP-3 is a potent inhibitor of the respiratory inflammation and airway hyperresponsiveness associated with obstructive respiratory disorders such as bronchial asthma. The GGG-IGFBP-3 mutant has nearly the same inhibitory effectiveness as the wild-type protein, demonstrating that inhibition is the result of intrinsic IGFBP-3 anti-inflammatory activity rather than merely the ability to block IGF activity. The results described herein indicate that alterations in IGFBP-3 levels are implicated in the pathogenesis of bronchial asthma and other obstructive respiratory disorders, and that restoration of IGFBP-3 will serve to prevent and suppress these disorders.

IGFBP-3 levels may be modulated to treat a subject suffering from or at risk for an obstructive respiratory disorder associated with inflammation of the respiratory tissue or airway hyperresponsiveness. IGFBP-3 levels in a subject may be increased by administering an exogenous IGFBP-3 polypeptide or an analog thereof, or a vector containing a nucleotide sequence encoding IGFBP-3 or an IGFBP-3 analog. Likewise, IGFBP-3 levels in a subject may be modulated by administering an agent or agents that modulate endogenous IGFBP-3 expression (see, e.g., U.S. Pat. No. 5,840,673).

Polypeptides, vectors, or other agents may be administered to a subject via any effective route known in the art, including for example aerosol, enteral, nasal, ophthalmic, oral, parenteral, or transdermal administration, and may be administered in conjunction with a pharmaceutically acceptable carrier.

The level of IGFBP-3 expression in a subject may be used to diagnose an obstructive respiratory disorder or a predisposition for developing such a disorder. Expression may be detected or measured in a fluid or tissue sample obtained from the subject and compared to expression levels from known healthy tissue. Decreased IGFBP-3 expression in the test sample may indicate that the subject suffers from or is risk for developing an obstructive respiratory disorder. IGFBP-3 expression may be measured over time in a subject known to suffer from or be at risk for an obstructive respiratory disorder. This will allow the subject to monitor the progression of the disorder and anticipate impending attacks. Measurement of IGFBP-3 expression levels over time may also be used to monitor the effectiveness of a therapeutic strategy or to customize and adjust a therapeutic approach for a subject.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Statistics

Data in the following examples were expressed as mean ±SEM. Statistical comparisons were performed using one-way ANOVA followed by the Fisher's test. Significant differences between groups were determined using the unpaired Student's t test. Statistical significance was set at P<0.05.

### Example 1

### Generation of WT-AdIGFBP-3, m-AdIGFBP-3 Vectors, and AdLacZ Vectors

Three adenovirus (Ad) vectors were created: WT-AdIGFBP-3, which contained IGFBP-3 cDNA; m-AdIGFBP-3, which contained GGG-IGFBP-3 cDNA; and AdLacZ, which was used as a control. The E1/E3-deleted replication-deficient recombinant Ad was made using the AdEasy system (He 1998) (Quantum Biotechnologies, Montreal, Quebec, Canada). NotI-XbaI restriction fragments from pcDNA3/wild-type IGFBP-3 and SalI-SmaI/EcoRV restriction fragments from GGG-IGFBP-3 mutant cDNAs were ligated into KpnI-XhoI-digested pShuttleCMV, as described previously (Kwak 2003). To create AdLacZ, a SalI-NotI restriction fragment from pcDNA3.1/LacZ (Invitrogen Corp., San Diego, Calif., USA) was ligated to SalI-NotI-digested pShuttleCMV. Recombination into the pAdEasy viral backbone was accomplished in bacteria (E. coli strain BJ5183, which is recombination deficient) according to the manufacturer's instructions. The recombination was verified and the adenoviral recombinant DNA was transferred to a regular strain of E. coli (DH5α), which generates far greater yields of DNA. Recombinant pAdEasy plasmids containing CMV-cDNA inserts were purified over QIAGEN columns (QIAGEN Inc., Valencia, Calif., USA), and 5 μg of PacI-digested DNA was used to transfect QBI-293A cells using the calcium phosphate method (Promega Corp., Madison, Wis., USA). Cells were seeded at 2×10⁶ cells per 150-mm culture dish 24 hours prior to transfection. Lysis of transfected cells, indicating adenoviral growth, occurred within four days. Following amplification, lysates containing clonal recombinant Ad were prepared from 150-mm culture dishes and purified by CsCl gradient centrifugation. Recovered virus was aliquoted and stored at −20° C. in 5 mM Tris (pH 8.0) buffer containing 50 mM NaCl, 0.05% BSA, and 25% glycerol. Virus was titrated by serial dilution infection of QBI-293A cells and plaques were counted under an overlay of 0.3% agarose, 10% FBS, and 1×DMEM.

### Example 2

### Effect of WT-AdIGFBP-3 and m-AdIGFBP-3 Administration on Cell Counts in BAL Fluid

Female C57BL/6 mice, 8-10 weeks of age and free of murine specific pathogens, were obtained from the Korean Research Institute of Chemistry Technology (Daejon, Korea). The mice were housed throughout the experiments in a laminar flow cabinet and were maintained on standard laboratory chow ad libitum. All experimental animals used in this study were treated according to guidelines approved by the Institutional Animal Care and Use Committee of the Chonbuk National University Medical School.

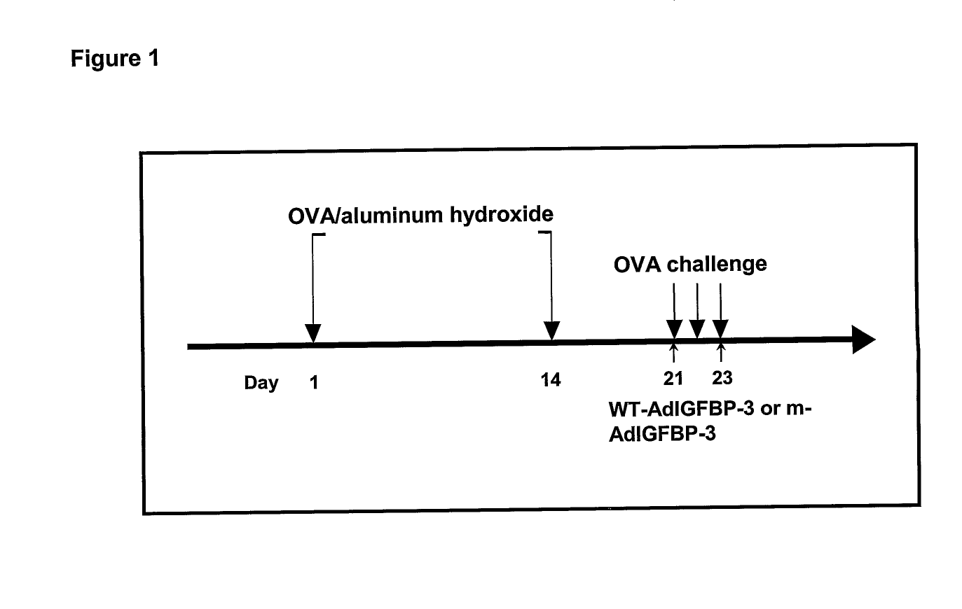

Mice were sensitized on days 1 and 14 by intraperitoneal injection of 20 μg ovalbumin (OVA)(Sigma-Aldrich, St. Louis, Mo., USA) emulsified in 1 mg of aluminum hydroxide (Pierce Chemical Co., Rockford, Ill., USA) in a total volume of 200 μl. Following the initial sensitization, mice were challenged on days 21, 22, and 23 with an aerosol of 3% (wt/vol) OVA in saline using an ultrasonic nebulizer (NE-U12; Omron Corp., Tokyo, Japan) for 30 minutes per day. Control mice received saline in place of OVA. Ad vectors (10⁹ plague-forming units) were administered intratracheally on day 21 (one hour prior to airway challenge with OVA) and day 23 (three hours after airway challenge). Control mice were administered with saline. A schematic of the administration protocol is shown in FIG. 1. This protocol resulted in five experimental groups: SAL+SAL, OVA+SAL, OVA+AdWT-IGFBP-3, OVA+m-AdIGFBP-3, and OVA+AdLacZ.

Bronchoalveolar lavage (BAL) was performed 72 hours after the last airway challenge on six mice from each experimental group. At the time of lavage, the mice were sacrificed with an overdose of sodium pentobarbitone (pentobarbital sodium, 100 mg/kg body weight, administered intraperitoneally). The chest cavity was exposed to allow for expansion, after which the trachea was carefully intubated and the catheter secured with ligatures. Prewarmed 0.9% NaCl solution was slowly infused into the lungs and withdrawn. BAL aliquots were pooled and stored at 4° C. Part of each pool was then centrifuged and the supernatants were stored at −70° C. until use.

Total cell numbers were counted with a hemocytometer. Smears of BAL cells were prepared by cytospin (Shandon Scientific Ltd., Cheshire, United Kingdom). The smears were stained with Diff-Quik solution (Dade Diagnostics of Puerto Rico Inc., Aguada, Puerto Rico) in order to examine the cell differentials. Two independent, blinded investigators counted the cells using a microscope. Approximately 400 cells were counted in each of four different random locations. The variation in results between the investigators was less than 5%. The mean of the values from the two investigators was used for each cell count.

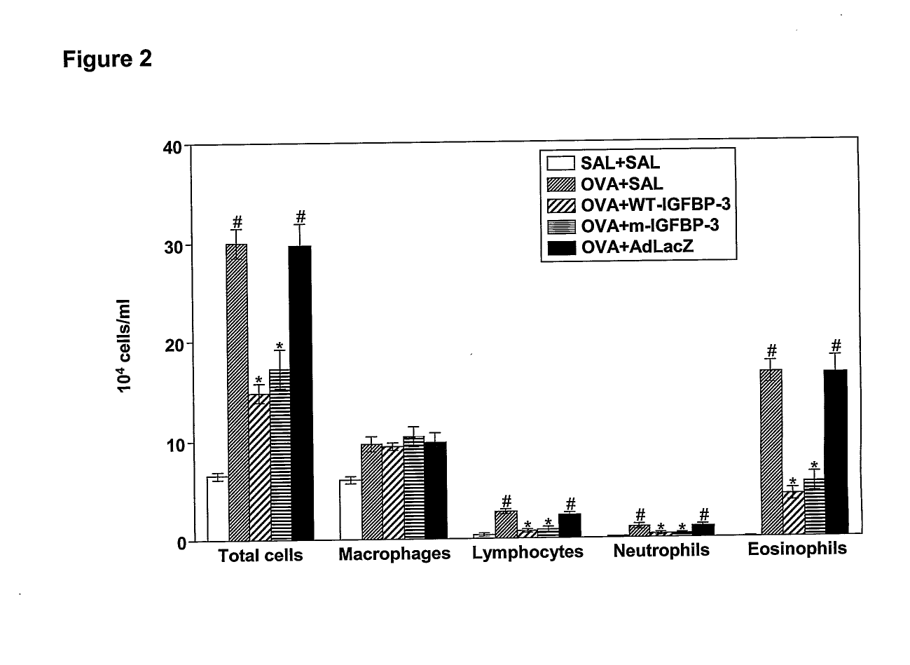

The number of total cells, eosinophils, lymphocytes, and neutrophils in BAL fluid was significantly increased at 72 hours after challenge with OVA (FIG. 2, compare “SAL+SAL” and “OVA+SAL”). The number of each cell type in OVA-challenged BAL fluid was significantly reduced by administration of WT-AdIGFBP-3 and m-AdIGFBP-3 (FIG. 2, “OVA+WT-IGFBP-3” and OVA+m-IGFBP-3”). Administration of AdLacZ had no effect on cell number.

### Example 3

### Effect of WT-AdIGFBP-3 and m-IGFBP-3 Administration on OVA-Induced Asthma Pathology

Following the BAL procedure described in Example 2, lungs were removed from the mice. Prior to removal, the lungs and trachea were filled intratracheally with a fixative (0.8% formalin, 4% acetic acid) using a ligature around the trachea. Lung tissues were fixed with 10% (v/v) neutral buffered formalin. The specimens were dehydrated and embedded in paraffin. For histological examination, 4 μm sections of fixed embedded tissues were cut on a Leica model 2165 rotary microtome (Leica, Nussloch, Germany), placed on glass slides, deparaffinized, and stained sequentially with hematoxylin 2 and eosin-Y (Richard-Allan Scientific, Kalamazoo, Mich.).

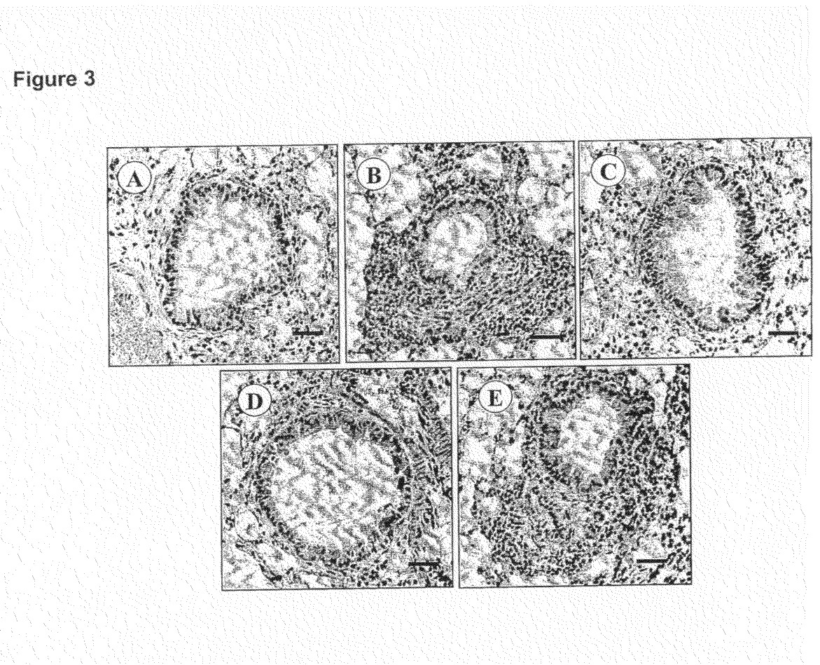

The histological examination revealed typical pathological features of asthma in the OVA-exposed mice. Numerous inflammatory cells, including eosinophils, infiltrated around the bronchioles in response to challenge with OVA (FIG. 3, compare panel A (SAL+SAL) and panel B (OVA+SAL)). Administration of WT-AdIGFBP-3 and m-AdIGFBP-3 resulted in a marked reduction in inflammatory cell infiltration in the peribronchiolar and perivascular regions of OVA-challenged mice (FIG. 3, panels C (OVA+WT-AdIGFBP-3) and D (OVA+m-AdIGFBP-3)). Administration of AdLacZ had no effect (FIG. 3E).

Each of the histological specimens was assigned an inflammation score by three independent blinded investigators. The degree of peribronchial and perivascular inflammation was evaluated on a subjective scale of 0 to 3, as described elsewhere (Tournoy 2000). A value of 0 was assigned when there was no detectable inflammation, a value of 1 was assigned when there was occasional cuffing with inflammatory cells, a value of 2 was assigned when most bronchi or vessels were surrounded by a thin layer (one to five cells) of inflammatory cells, and a value of 3 was assigned when most bronchi or vessels were surrounded by a thick layer (more than five cells) of inflammatory cells.

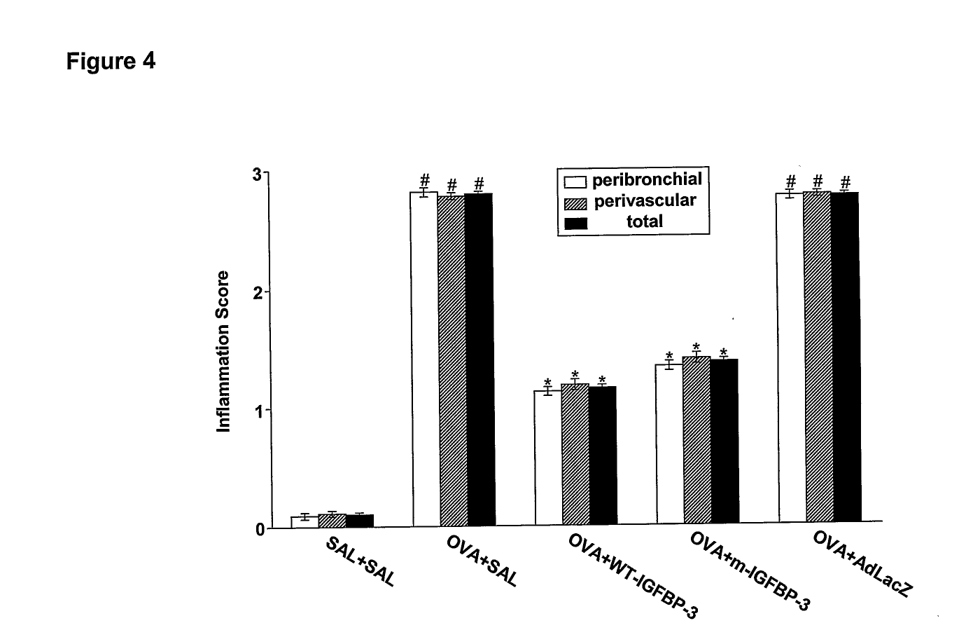

Peribronchial, perivascular, and total lung inflammation scores were increased significantly in specimens challenged with OVA (FIG. 4, compare “SAL+SAL” and “OVA+SAL”). All three scores were significantly reduced in OVA-challenged specimens that had been administered WT-AdIGFBP-3 or m-AdIGFBP-3 (FIG. 4, “OVA+WT-IGFBP-3” and “OVA+mIGFBP-3”). These results suggest that WT-AdIGFBP-3 and m-AdIGFBP-3 inhibit antigen-induced inflammation in the lungs, including the influx of eosinophils.

### Example 4

### Effect of WT-AdIGFBP-3 and m-AdIGFBP-3 Administration on IL-4, IL-5, and IL-13 Expression

Lung tissues were homogenized in the presence of protease inhibitors to obtain extracts of proteins. Protein concentrations were determined using the Bradford reagent (Bio-Rad). Samples (30 μg of protein per lane) were loaded on a 12% SDS-PAGE gel. After electrophoresis at 120 V for 90 minutes, separated proteins were transferred to polyvinylidene difluoride membranes (Amersham Pharmacia Biotech, Piscataway, N.J.) by the wet transfer method (250 mA, 90 minutes). Nonspecific sites were blocked with 5% non-fat dry milk in TBST buffer (25 mM Tris, pH 7.5, 150 mM NaCl, 0.1% Tween 20) for 1 hour, after which the blots were incubated with anti-IL-4 antibody (Serotec Ltd, Oxford, UK), anti-IL-5 antibody, or anti-IL-13 antibody (R&D Systems, Inc. Minneapolis, Minn.) overnight at 4° C. Anti-rabbit horseradish peroxidase conjugated IgG was used to detect binding of antibodies. The membranes were stripped and reblotted with anti-actin antibody (Sigma-Aldrich) to verify equal loading of protein in each lane. The binding of the specific antibodies was visualized by exposing to photographic film after treatment with enhanced chemiluminescence system reagents (Amersham Pharmacia Biotech).

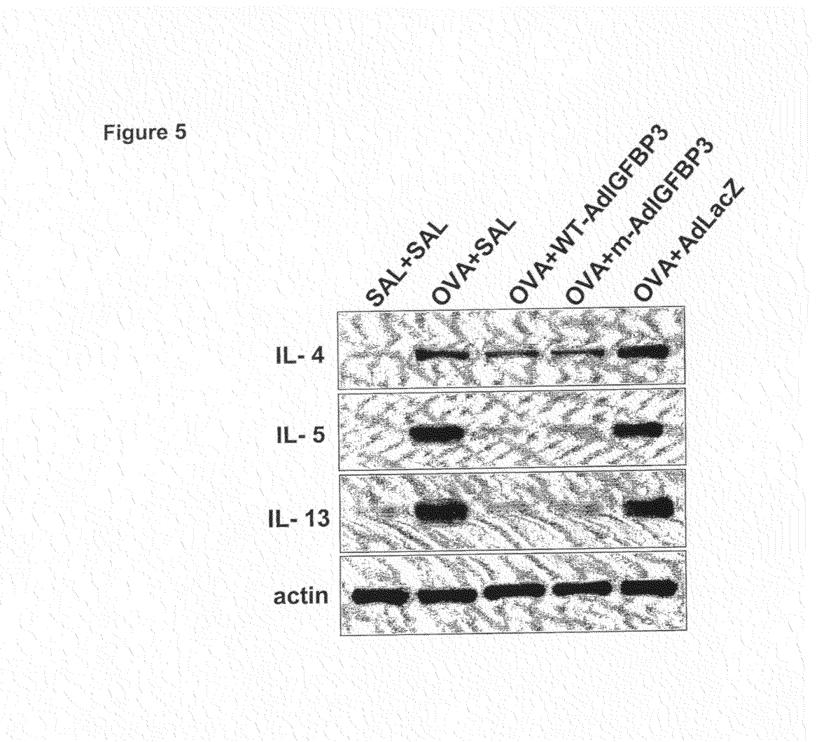

Western blot analysis revealed that IL-4, IL-5, and IL-13 cytokine levels in lung tissue were significantly increased following challenge with OVA (FIG. 5, compare “SAL+SAL” and “OVA+SAL”). The level of each cytokine in OVA-challenged tissue was markedly reduced by administration of WT-AdIGFBP-3 or m-AdIGFBP-3 (FIG. 5, “OVA+WT-AdIGFBP3” and “OVA+m-AdIGFBP3”).

IL-4, IL-5, and IL-13 levels were quantified in the supernatants of BAL fluids by enzyme immunoassays according to the manufacturer's protocol (IL-4 and IL-5, Endogen, Inc., Woburn, Mass., USA; IL-13, R&D Systems, Inc., Minneapolis, Minn., USA). Sensitivities for IL-4, IL-5, and IL-13 assays were 5, 5, and 1.5 μg/ml, respectively.

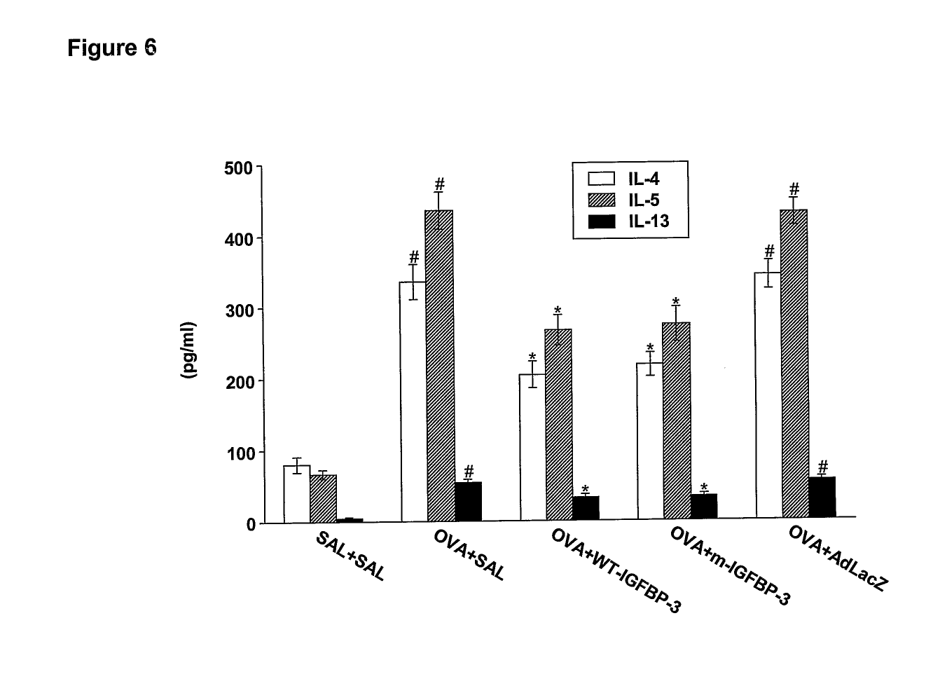

The enzyme immunoassays were consistent with the results obtained from the Western blot analysis. IL-4, IL-5, and IL-13 levels were increased in BAL fluid from OVA-challenged subjects (FIG. 6, compare “SAL+SAL” and “OVA+SAL”), and this increase was significantly reduced by administration of WT-AdIGFBP-3 or m-AdIGFBP-3 (FIG. 6, “OVA+WT-IGFBP-3” and “OVA+m-IGFBP-3”).

### Example 5

### Effect of WT-AdIGFBP-3 and m-AdIGFBP-3 Administration on TNF-α and IL-1β Expression

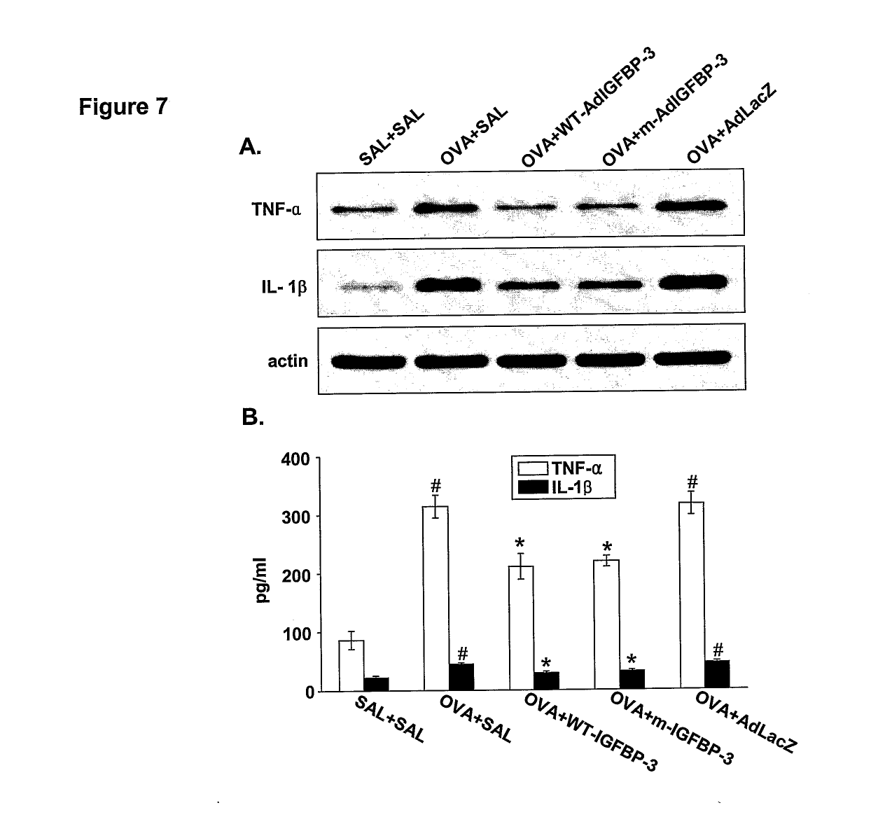

TNF-α and IL-1β, expression levels in lung tissue were measured by Western blotting as described in Example 4. Levels of both proteins were significantly increased at 72 hours after challenge with OVA (FIG. 7A, compare “SAL+SAL” and “OVA+SAL”). The level of both proteins in OVA-challenged tissue was significantly reduced by administration of WT-AdIGFBP-3 or m-AdIGFBP-3 (FIG. 7A, “OVA+WT-AdIGFBP-3” and “OVA+m-AdIGFBP-3”).

TNF-α and IL-1β expression levels in BAL fluid were measured by enzyme immunoassays as described in Example 4. Consistent with the results of the Western blot analysis, TNF-α and IL-1β expression was increased following challenge with OVA (FIG. 7B, “SAL+SAL” and “OVA+SAL”), and this increase was significantly reduced by administration of WT-AdIGFBP-3 or m-AdIGFBP-3 (FIG. 7B, “OVA+WT-IGFBP-3” and “OVA+m-IGFBP-3”).

### Example 6

### Effect of WT-AdIGFBP-3 and m-AdIGFBP-3 Administration on VCAM-1 and ICAM-1 Expression

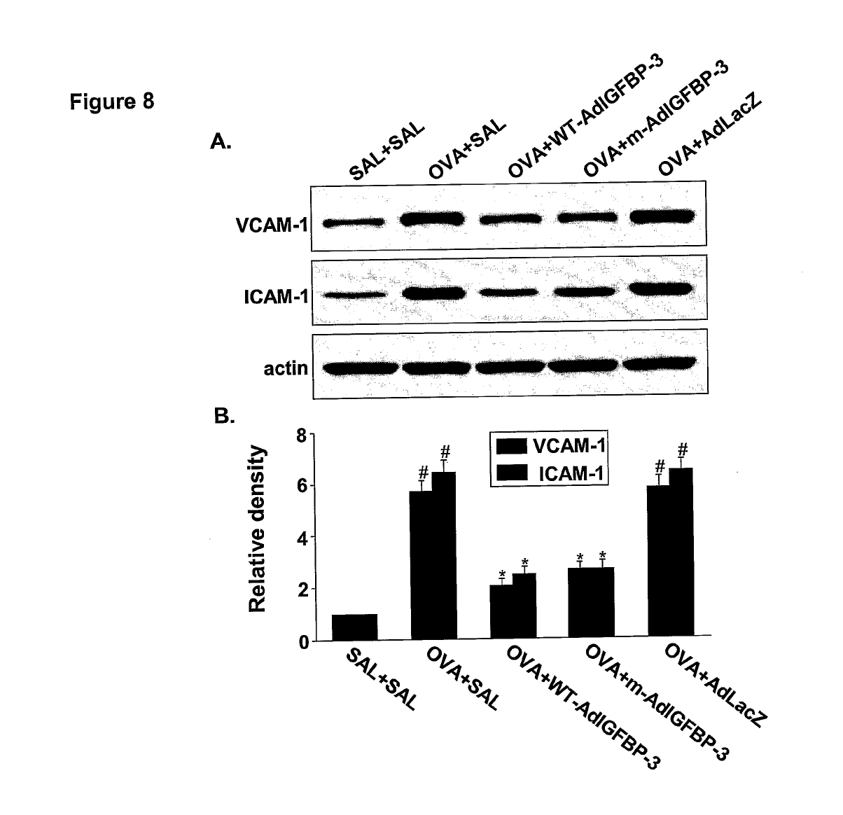

Vascular cell adhesion molecule-1 (VCAM-1) and intercellular adhesion molecule-1 (ICAM-1) expression levels in lung tissue were measured by Western blotting as described in Example 4, and quantitated by densitometric analysis. Levels of both proteins were significantly increased at 72 hours after challenge with OVA (FIGS. 8A and 8B, compare “SAL+SAL” and “OVA+SAL”). The level of both proteins in OVA-challenged tissue was significantly reduced by administration of WT-AdIGFBP-3 or m-AdIGFBP-3 (FIGS. 8A and 8B, “OVA+WT-AdIGFBP-3” and “OVA+m-AdIGFBP-3”).

### Example 7

### Effect of WT-AdIGFBP-3 and m-AdIGFBP-3 Administration on Eotaxin and RANTES Expression

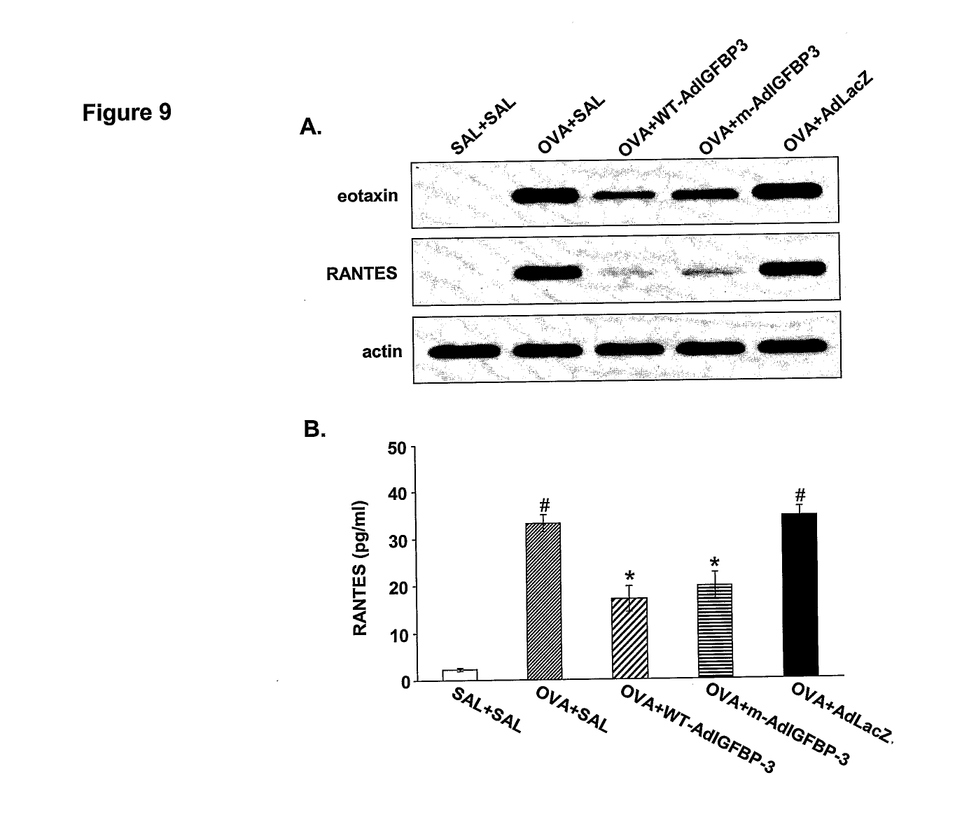

Eotaxin and RANTES expression levels in lung tissue were measured by Western blotting as described in Example 4. Levels of both proteins were significantly increased at 72 hours after challenge with OVA (FIG. 9A, compare “SAL+SAL” and “OVA+SAL”). The level of both proteins in OVA-challenged tissue was significantly reduced by administration of WT-AdIGFBP-3 or m-AdIGFBP-3 (FIG. 9A, “OVA+WT-AdIGFBP3” and “OVA+m-AdIGFBP3”).

Eotaxin and RANTES expression levels in BAL fluid were measured by enzyme immunoassays as described in Example 4. Consistent with the results of the Western blot analysis, eotaxin and RANTES expression was increased following challenge with OVA (FIG. 9B, “SAL+SAL” and “OVA+SAL”), and this increase was significantly reduced by administration of WT-AdIGFBP-3 or m-AdIGFBP-3 (FIG. 9B, “OVA+WT-IGFBP-3” and “OVA+m-IGFBP-3”).

### Example 8

### Effect of WT-AdIGFBP-3 and m-AdIGFBP-3 Administration on Airway Hyperresponsiveness

Airway responsiveness was measured in mice three days after the last challenge in an unrestrained conscious state, as previously described (Lee 2002). Mice were placed in a barometric plethysmographic chamber (All Medicus Co., Seoul, Korea) and baseline readings were taken and averaged for three minutes. Aerosolized methacholine in increasing concentrations (2.5 to 50 mg/ml) was nebulized through an inlet of the main chamber for three minutes at a time. Readings were taken and averaged for three minutes after each nebulization. Enhanced pause (Penh, calculated as (expiratory time/relaxation time-1)×(peak expiratory flow/peak inspiratory flow), according to the manufacturers' protocol) is a dimensionless value that represents a function of the proportion of maximal expiratory to maximal inspiratory box pressure signals and a function of the timing of expiration. The percent increase in baseline Penh was used to assess airway responsiveness at increasing methacholine concentrations, with the baseline Penh (after challenge with saline) expressed as 100%.

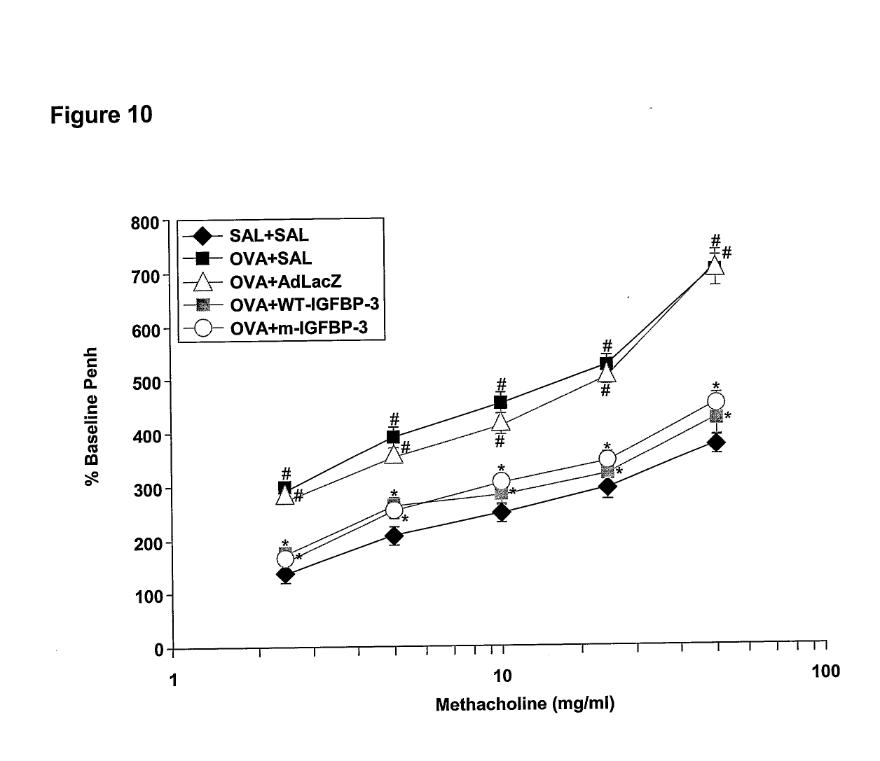

The dose-response curve of percent baseline Penh shifted to the left in mice challenged with OVA (FIG. 10, compare “SAL+SAL” and “OVA+SAL”), indicating airway hyperresponsiveness following OVA challenge. Administration of increasing concentrations of methacholine increased the percent baseline Penh in both OVA-challenged mice and control mice, with the OVA-challenged mice exhibiting a higher percent baseline Penh than control mice at each methacholine concentration tested (FIG. 10, compare “SAL+SAL” and “OVA+SAL”). Administration of WT-AdIGFBP-3 or m-AdIGFBP-3 to OVA-challenged mice shifted the dose-response curve of percent baseline Penh to the right compared to untreated OVA-challenged mice (FIG. 10, compare “OVA+SAL,” OVA+WT-IGFBP-3,” and “OVA+m-IGFBP-3”).

### Example 9

### IGFBP-3 Expression in Normal and OVA-Challenged Lung Tissue

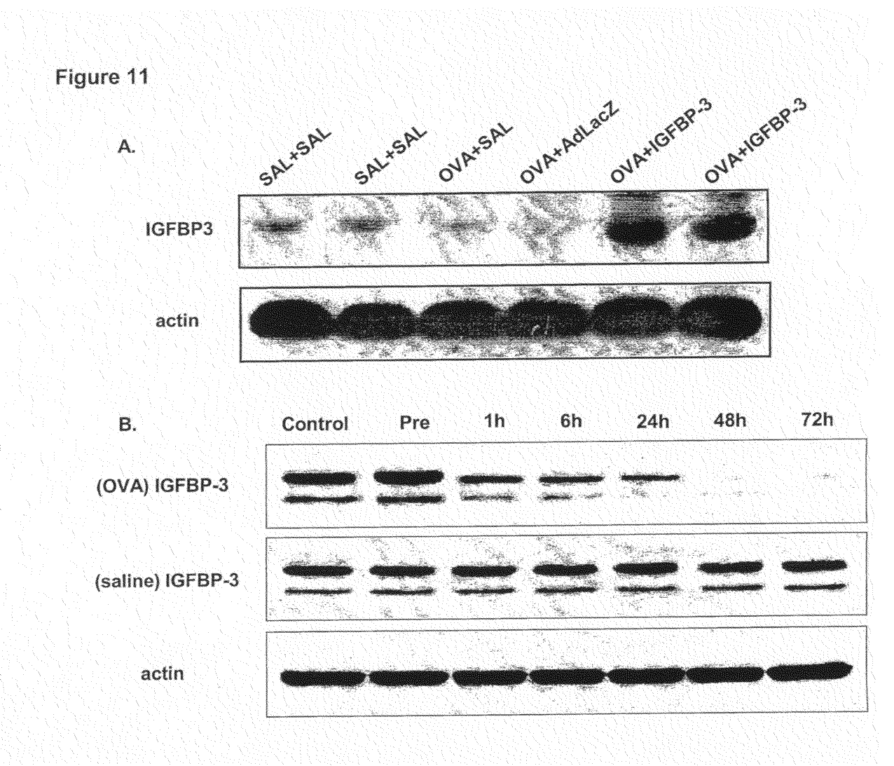

Western blot analysis was performed on lung protein extract as described in Example 4 using IGFBP-3 antibody (Santa Cruz Biotechnology, Santa Cruz, Calif.). Endogenous IGFBP-3 expression was significantly reduced in OVA-challenged lung tissue compared to normal lung tissue (FIG. 11a, compare “SAL+SAL” and “OVA+SAL”). IGFBP-3 levels decreased over time following OVA challenge, but did not show any significant change in control cells (FIG. 11b, compare “(OVA) IGFBP-3” and “(saline) IGFBP-3” at various timepoints). Increased expression of IGFBP-3 was observed in mice administered AdIGFBP-3, validating the adenoviral gene transfer technique (FIG. 11a, “OVA+IGFBP-3”).

### Example 10

### IGF-1 Expression in Normal and OVA-Challenged Lung Tissue

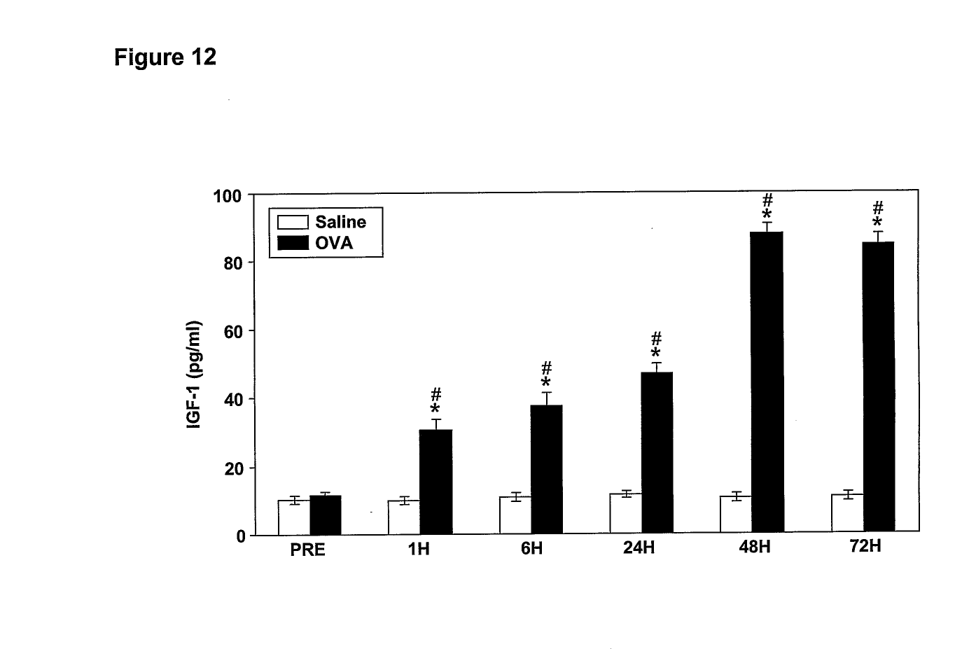

IGF-1 expression levels in BAL fluid were measured by enzyme immunoassay as described in Example 4. Levels of IGF-1 were significantly increased at 1 hour, 6 hours, 24 hours, 48 hours, and 72 hours after challenge with OVA (FIG. 12A, compare “PRE” to “1H,” 6H,” “24H,” “48H,” and “72H”). No significant change in IGF-1 levels was observed after saline inhalation.

### Example 11

### Localization of Immunoreactive IGFBP-3 in Lung Tissue and Tracheal Epithelial Cells

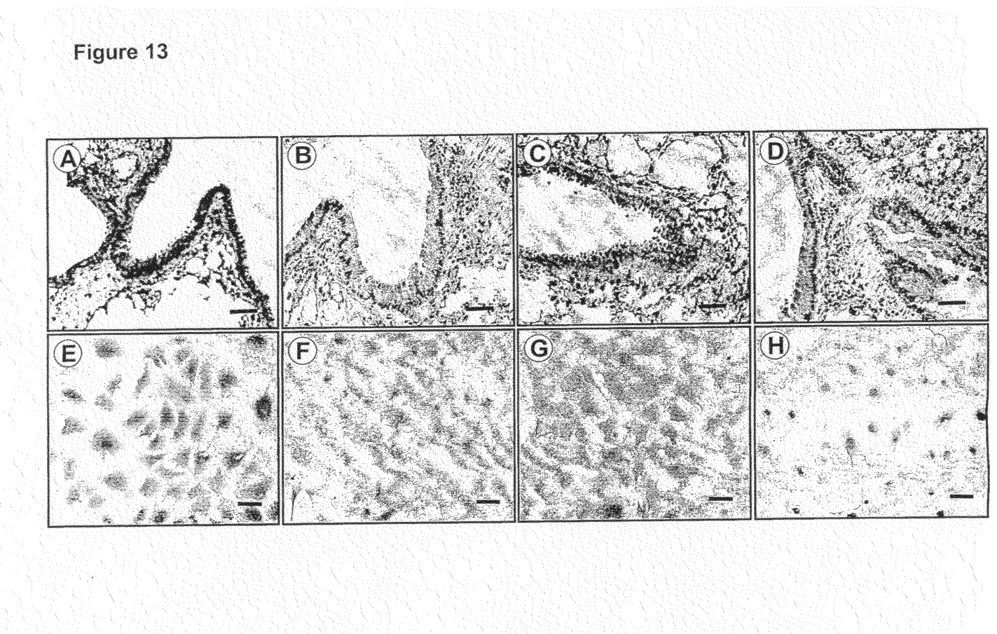

The localization of IGFBP-3 in lung tissue was observed by histological analysis. IGFBP-3 was localized in epithelial layers around the bronchioles of control mice, but disappeared in mice challenged with OVA (FIG. 13, compare A and B). Intratracheal administration of WT-AdIGFBP-3 restored IGFBP-3 expression in the lung tissue of OVA-challenged mice, whereas administration of AdLacZ had no effect (FIG. 13, compare C and D).

The localization of IGFBP-3 in tracheal epithelial cells was observed by histological analysis. Mice tracheal epithelial cells were isolated under sterile conditions. The trachea proximal to the bronchial bifurcation was excised, and adherent adipose tissue was removed. The trachea was opened longitudinally, cut into three pieces, and incubated in DMEM containing 0.1% protease overnight at 4° C. Following tissue digestion, FBS (10% final) was added to the medium to deactivate enzymes. Undigested tissue fragments were removed, and tracheal epithelial cells were harvested by centrifugation at 500 rpm for 5 minutes. The cells were seeded onto collagen-coated 35 mm dishes for submerged culture. The growth medium DMEM/F-12 (Sigma-Aldrich), which contains 10% FBS, penicillin, streptomycin, and amphotericin B was supplemented with insulin, transferrin, hydrocortisone, phosphoethanolamine, cholera toxin, ethanolamine, bovine pituitary extract, and BSA. Cells were maintained in a humidified 5% CO₂ incubator at 37° C. until they adhered.

IGFBP-3 was localized in tracheal epithelial cells of control mice, but markedly reduced in mice challenged with OVA (FIG. 13, compare E and F). Intratracheal administration of WT-AdIGFBP-3 restored IGFBP-3 expression in the lung tissue of OVA-challenged mice, whereas administration of AdLacZ had no effect (FIG. 13, compare G and H).

### Example 12

### Effect of Recombinant IGFBP-3 Administration on Cell Counts in BAL Fluid

Mice were sensitized on days 1 and 14 by intraperitoneal injection of 20 μg OVA emulsified in 1 mg of aluminum hydroxide in a total volume of 200 μl. Following the initial sensitization, mice were challenged on days 21, 22, and 23 with an aerosol of 3% (wt/vol) OVA in saline using an ultrasonic nebulizer (NE-U12) for 30 minutes per day. Control mice received saline in place of OVA. Recombinant IGFBP-3 was administered on day 21 (one hour prior to airway challenge with OVA) and day 23 (three hours after airway challenge). Control mice were administered with saline. This protocol resulted in four experimental groups: SAL+SAL, OVA+SAL, OVA+IGFBP-3 μg, and OVA+IGFBP3 10 μg.

BAL was performed as described in Example 2 at 72 hours after the last airway challenge on ten mice from each experimental group. BAL smears were examined by two independent, blinded investigators using a microscope. The mean of the values from the two investigators was used for each cell count.

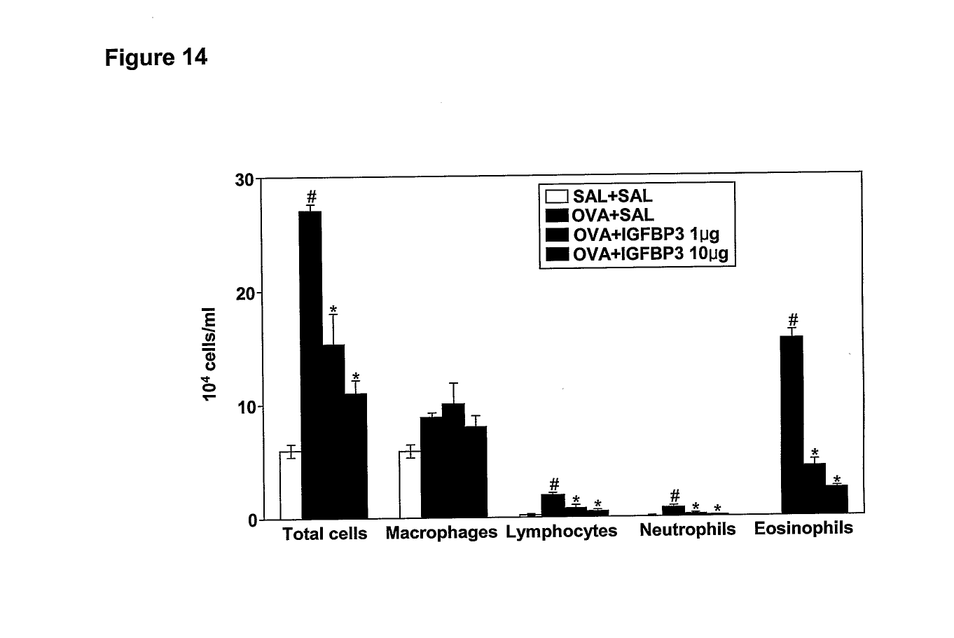

The number of total cells, eosinophils, lymphocytes, and neutrophils in BAL fluids was significantly increased at 72 hours after challenge with OVA (FIG. 14, compare “SAL+SAL” and “OVA+SAL”). Administration of 1 μg recombinant IGFBP-3 resulted in a decrease in total cells, lymphocytes, neutrophils, and eosinophils (FIG. 14, “OVA+IGFBP3 1 μg”). Administration of 10 μg recombinant IGFBP-3 resulted in an even greater decrease in each of these cell types, and also led to a decrease in the number of macrophages (FIG. 14, “OVA+IGFBP3 10 μg”).

### Example 13

### Effect of Recombinant IGFBP-3 on Airway Hyperresponsiveness

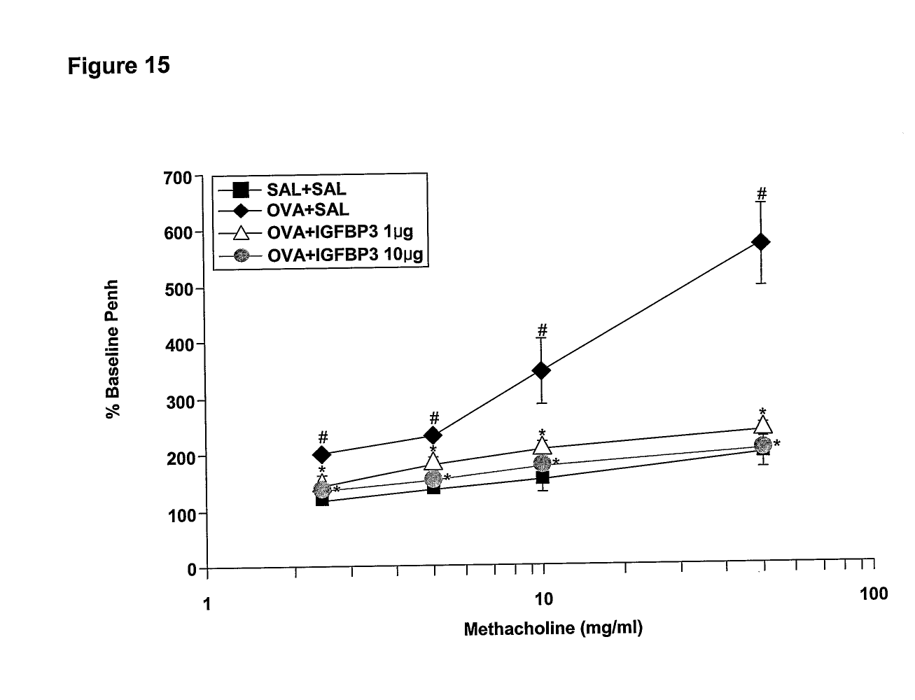

Airway responsiveness was measured in mice from Example 12 three days after the last challenge as described in Example 8. The dose-response curve of percent baseline Penh shifted to the left in mice challenged with OVA (FIG. 15, compare “SAL+SAL” and “OVA+SAL”), indicating airway hyperresponsiveness following OVA challenge. In addition, the increase in the percent baseline Penh in response to increasing methacholine concentrations was greater in the mice challenged with OVA. Administration of recombinant IGFBP-3 reduced airway hyperresponsiveness, as indicated by a shift in the dose-response curve of percent baseline Penh to the right (FIG. 15, compare “OVA+SAL,” OVA+IGFBP3 μg,” and “OVA+IGFBP3 10 μg).

As stated above, the foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein.

### REFERENCES

1. Bach, L. A., Rechler, M. M. 1995. Insulin-like growth factor binding proteins. Diabetes Rev 3:38-61.
2. Baxter, R. C. 1991. Insulin-like growth factor (IGF) binding proteins: the role of serum IGFBPs in regulating IGF availability. Acta Paediatr Scand Suppl 372:107-114.
3. Buckway, C. K., et al. 2001. Mutation of three critical amino acids of the N-terminal domain of insulin-like growth factor (IGF) binding protein-3 essential for high-affinity IGF binding. J Clin Endocrinol Metab 86:4943-4950.
4. Cambrey, A. D., et al. 1995. Insulin-like growth factor 1 is a major mitogen produced by primary cultures of human airway epithelial cells. Clin Sci (Lond) 89:611-617.
5. Edmondson, S. R., et al. 2003. Epidermal homeostasis: the role of the growth hormone and insulin-like growth factor systems. Endocr Rev 24:737-764.
6. Frigas, E., Gleich, G. J. 1986. The eosinophil and the pathophysiology of asthma. J Allergy Clin Immunol 77:527-537.
7. He, T. C., et al. 1998. A simplified system for generating recombinant adenoviruses. Proc Natl Acad Sci USA 95:2509-2514.
8. Holgate, S. T., et al. 2000. Epithelial-mesenchymal interactions in the pathogenesis of asthma. J Allergy Clin Immunol 105:193-204.
9. Hoshino, M., et al. 1998. Inhaled corticosteroid reduced lamina reticularis of the basement membrane by modulation of insulin-like growth factor (IGF-1)-I expression in bronchial asthma. Clin Exp Allergy 28:568-577.
10. Jones, J. I., et al. 1995. Insulin-like growth factors and their binding proteins: biological actions. Endocr Rev 16:3-34.
11. Kay, A. B. 1991. Asthma and inflammation. J Allergy Clin Immunol 87:893-910.
12. Kim, H-S., et al. 2004. Insulin-like growth factor binding protein-3 induces apoptosis through the activation of caspases involved in a death receptor-mediated pathway in MCF-7 human breast cancer cells. Cancer Res 64:2229-2237.
13. Kwak, Y. G., et al. 2003. Involvement of PTEN in airway hyperresponsiveness and inflammation in bronchial asthma. J Clin Invest 111:1083-1092.
14. Lee, Y. C., Kwak, Y. G., Song, C. H. 2002. Contribution of vascular endothelial growth factor to airway hyper-responsiveness and inflammation in a murine model of toluene diisocyanate-induced asthma. J Immunol 168:3595-3600.
15. Le Roith, D., et al. 2001. The somatomedin hypothesis. Endocr Rev 22:53-74.
16. Longobardi, L., et al. 2003. A novel insulin-like growth factor (IGF)-independent role for IGF binding protein-3 in mesenchymal chondroprogenitor cell apoptosis. Endocrinology 144:1695-1702.
17. Nissley, P., Lopaczynski, W. 1991. Insulin-like growth factor receptors. Growth Factors 5:29-43.
18. O'Byrne, M. B., Inman, M. D. 2003. Airway hyperresponsiveness. Chest 123:411 S-416S.
19. Oh, Y., et al. 1993. Insulin-like growth factor (IGF)-independent action of iGF-binding protein-3 in Hs579T human breast cancer cells. J Biol Chem 268:14964-14971.
20. Oh, Y., et al. 1998. IGF-independent actions of IGFBPs. Molecular Mechanisms to Regulate the Activities of Insulin-like Growth Factors, Takano, K., et al., eds., Elsevier, Amsterdam, pp. 123-133.
21. Rajah, R., et al. 1999. Elevated levels of the IGF-binding protein protease MMP-1 in asthmatic airway smooth muscle. Am J Respir Cell Mol Biol 20:199-208.
22. Rinderknecht, E., Humbel, R. E. 1978a. The amino acid sequence of human insulin-like growth factor I and its structural homology with proinsulin. J Biol Chem 253:2769-2776.
23. Rinderknecht, E., Humbel, R. E. 1978b. Primary structure of human insulin-like growth factor II. FEBS Lett 89:283-286.
24. Rosenfeld, R., et al. 1986. Insulin-like growth factors I and II in the evaluation of growth retardation. J Pediatr 109:428-433.
25. Rotwein, P. 1991. Structure, evolution, expression and regulation of insulin-like growth factors I and II. Growth Factors 5:3-18.
26. Schumacher, R., et al. 1991. Insulin and insulin-like growth factor-I binding specificity is determined by distinct regions of their cognate receptors. J Biol Chem 266:19288-19295.
27. Shimasaki, S., Ling, N. 1991. Identification and molecular characterization of insulin-like growth factor binding proteins (IGFBP-1, -2, -3, -4, -5 and -6). Prog Growth Factor Res 3:243-266.
28. Tournoy, K. G., et al. 2000. Airway eosinophilia is not a requirement for allergen-induced airway hyperresponsiveness. Clin Exp Allergy 30:79-85.

1. A method of treating a condition associated with airway hyperresponsiveness in a subject comprising administering a vector containing a nucleotide sequence encoding IGFBP-3 or an analog thereof.

2. The method of claim 1, wherein said analog is GGG-IGFBP-3.

3. The method of claim 1, wherein said vector is an adenovirus.

4. The method of claim 1, wherein said condition is asthma.

5-7. (canceled)

8. A method of treating an obstructive respiratory disorder comprising administering a vector containing a nucleotide sequence encoding IGFBP-3 or an analog thereof.

9. The method of claim 8, wherein said analog is GGG-IGFBP-3.

10. The method of claim 8, wherein said vector is an adenovirus.

11. The method of claim 8, wherein said condition is asthma.

12. A method of decreasing inflammation in lower respiratory tissue comprising administering a vector containing a nucleotide sequence encoding IGFBP-3 or an analog thereof.

13. The method of claim 12, wherein said analog is GGG-IGFBP-3.

14. The method of claim 12, wherein said vector is an adenovirus.

15. The method of claim 12, wherein said condition is asthma.

16. A method of diagnosing a subject with a condition associated with airway hyperresponsiveness or with a predisposition for a condition associated with airway hyperresponsiveness, comprising detecting the expression level of IGFBP-3 in the subject relative to the expression level of IGFBP-3 in a normal subject.

17. A method of treating or preventing an antigen-induced pathological condition in a subject, comprising applying to lung tissue of the subject an exogenous IGFBP-3 polypeptide or analog thereof in an amount effective to treat or prevent the antigen-induced pathological condition in lung tissue of the subject, wherein the antigen-induced pathological condition is antigen-induced airway hyperresponsiveness, antigen-induced inflammation in lung tissue, antigen-induced influx of eosinophils in lung tissue, or antigen-induced increase in the level of a factor in lung tissue, and wherein the factor is IL-1β, IL-4, IL-5, IL-13, TNF-α, VCAM-1, ICAM-1, esotaxin, or RANTES.

18. The method of claim 17, wherein the analog is GGG-IGFBP-3.

19. A method of treating or preventing an antigen-induced pathological condition in a subject, comprising applying to the subject an agent in an amount effective to increase endogenous IGFBP-3 production in lung tissue of the subject, wherein the antigen-induced pathological condition is antigen-induced airway hyperresponsiveness, antigen-induced inflammation in lung tissue, antigen-induced influx of eosinophils in lung tissue, or antigen-induced increase in the level of a factor in lung tissue, and wherein the factor is IL-1β, IL-4, IL-5, IL-13, TNF-α, VCAM-1, ICAM-1, esotaxin, or RANTES.
